(19) United States
(12) Patent Application Publication
(10) Pub. No.: US 2013/0078283 A1
(43) Pub. Date: Mar. 28, 2013

(54) NOVEL AMPHIPHILES

(71) Applicants: Yi-Yan Yang, Singapore (SG); Shrinivas Venkataraman, Singapore (SG)

(72) Inventors: Yi-Yan Yang, Singapore (SG); Shrinivas Venkataraman, Singapore (SG)

(21) Appl. No.: 13/521,030

(22) PCT Filed: Jan. 6, 2011
(86) PCT No.: PCT/SG2011/000007

§ 371 Date: Nov. 12, 2012

(30) Foreign Application Priority Data
Jan. 6, 2010 (SG) 201000058-6

(51) Int. Cl. C07C 335/16; A61K 9/107; A61K 47/18; A61K 9/10; C07C 275/28; A61K 47/20

(52) U.S. Cl. CPC C07C 335/16; C07C 275/28; A61K 47/20; A61K 47/18; A61K 9/10; A61K 9/107
USPC 424/400; 564/28; 564/27; 564/50; 514/788

(57) ABSTRACT

Disclosed herein is a compound of structure (A): In this compound, X is either O or S, R¹ is a rigid group, R² is a hydrophilic group such that (A) is capable of self-assembly in water, and R³ is an organic group.

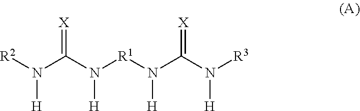

### TECHNICAL FIELD

The present invention relates to novel amphiphiles and processes for making them.

### INCORPORATION BY CROSS-REFERENCE

This application claims priority from Singaporean patent application no. 201000058-6 filed on 6 Jan. 2010, the entire contents of which are incorporated herein by cross-reference.

### BACKGROUND OF THE INVENTION

Spontaneous molecular self-assembly occurs due to the interplay of associative and repulsive interactions amongst various components in macromolecules, leading to microphase separation, and thereby enabling formation of well defined discrete nanoscopic structures. Solution state self-assembly of amphiphiles can lead to well defined nanoscale structures that have significant biomedical applications. In appropriate size regime (≦200 nm), these nanostructures can be easily taken up by the cells, allowing for the enhanced therapy. If the nanostructures have a hydrophilic surface such as PEG, they can transport drugs with prolonged blood circulation and enhanced permeation and retention at leaky tumor tissues (passive targeting based on EPR effect). Such well defined nanostructures are promising for numerous classes of therapeutic compounds, including DNA, siRNA, proteins, peptides and small molecules.

In general, micelles and vesicles are two important kinds of self-assembled nanostructures that are routinely employed for the encapsulation of water insoluble and soluble classes of therapeutics respectively. Partitioning of water insoluble drugs in the hydrophobic compartments of micelles has received lot of attention as a potential strategy for numerous diseases including cancer. Often, for the water soluble class of therapeutics, protection from the opportunistic and unforgiving in vivo conditions is necessary. For such systems, encapsulation into a vesicle is promising as it can be implemented by simple dissolution in water containing therapeutics and/or nutrients and the vesicle acts as a protective barrier, and thereby enhancing the bioavailability of the enclosed compounds. For successful molecular design of the vesicles, careful engineering of the inter-molecular interactions is important.

Designing molecular components not only with amphiphicity but also other types of interactions such as hydrogen bonding can afford improved control over the formation of self-assembled nanostructures. Recently, synthetic strategies to regioselectively introduce hydrogen bonding units in the molecular design have received significant attention. A wide array of hydrogen-bonding motifs has been identified. Amongst them, urea and thiourea are widely applicable due to their strong interactions and relative ease of synthesis. Urea or thiourea based molecular recognition units have been employed for wide variety of problems involving self assembly such as, solid-state crystal engineering, self-healing rubber, thermoplastic elastomers, and hydrogels. Furthermore, such rigid molecular recognition units have implications in controlling the morphology of the nanostructures like elongated micelles and recently, nanotubes. Bolaamphiphiles, which are essentially a hydrophobic component with hydrophilic head on both the sides, offer numerous advantages including higher solubility, lower critical aggregation concentration (CAC), increased stability. Such amphiphiles are often found in nature, particularly in some bacterial cell wall membranes. Due to their regioselective placement of amphiphilicity and rigid hydrophobic component, these amphiphiles are ideally suited for curved lamellae, where in the curvature of the lamellae could potentially be controlled by varying the ratio of hydrophobic versus hydrophilic components.

### OBJECT OF THE INVENTION

It is the object of the present invention to provide novel amphiphiles, processes for making them and methods for using them.

### SUMMARY OF THE INVENTION

In a first aspect of the invention there is provided a compound of structure (A):

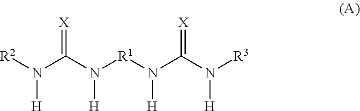

wherein:
X is either O or S;
R¹ is a rigid group;
R² is a hydrophilic group such that (A) is capable of self-assembly in water; and
R³ is an organic group.

The following options may be used in conjunction with the first aspect, either individually or in any suitable combination.

X may be S. X may be O.

At least one of R² and R³ may be oligomeric or polymeric. R² and R³ may both be oligomeric or polymeric. R² is commonly an organic group. It may comprise an ether linkage. It may comprise an oligoether or a polyether chain, e.g. an oligo- or poly-oxyethylene chain. In some embodiments, R² and R³ are the same. In other embodiments, R³ is a hydrophobic chain. In the latter embodiments, the hydrophobic chain may comprise (or may be) an aliphatic hydrocarbon chain. It may have a terminal methyl group. It may have a terminal hydroxyl group. It may have some other terminal group. In yet other embodiments R² and R³ are both hydrophilic (optionally both comprise ether groups, for example oligoether or polyether groups) but are not the same.

R¹ may comprise, or may be, an aromatic group, e.g. a carbocyclic aromatic group.

R¹, R² and R³ may be such that the compound has a critical aggregation concentration in water of below about 100 μM.

The compound (A) may be non-cytotoxic. It may be non-toxic towards HepG2 cells.

In an embodiment, X is S, R₁ is a carbocyclic aromatic group, e.g. 1,4-phenyl, and R² and R³ are the same and are oligo- or polyoxyethylene chains.

In another embodiment, X is O or S, R₁ is a rigid group, e.g comprising a carbocyclic aromatic group, R² is a polar group comprising an ether linkage and R³ is either the same as R² or is a hydrocarbon group having a terminal methyl or hydroxyl group.

In another embodiment, X is S, R¹ is a carbocyclic aromatic group, e.g. 1,4-phenyl, R² is an oligo- or polyoxyethylene chain and R³ is a hydrocarbon chain.

In another embodiment X is O, R¹ comprises a carbocyclic aromatic group, e.g. R¹ is methylenediphenyl-4,4′-diyl, and R² and R³ are the same and are oligo- or polyoxyethylene chains.

In another embodiment X is O, R¹ comprises a carbocyclic aromatic group, e.g. R¹ is methylenediphenyl-4,4′ diyl, R² is an oligo- or polyoxyethylene chain and R³ is a hydrocarbon chain.

In another embodiment, X is either O or S; R¹ is a rigid molecule, oligomer or polymer, preferably comprising at least one phenyl group; R² is an non-ionic oligomeric or polymeric moiety that is not an amine containing group; R³ is an non-ionic oligomeric or polymeric moiety that is not an amine containing group; and wherein at least one of R² and R³ is a hydrophilic moiety.

In a second aspect of the invention there is provided a process for making a compound of structure (A)

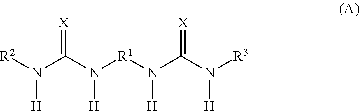

wherein:
X is either O or S;
R¹ is a rigid group;
R² is a hydrophilic group such that (A) is capable of self-assembly in water; and
R³ is an organic group,
said process comprising:
if R² and R³ are the same, reacting R¹(NCX)₂ with at about two mole equivalents of R²NH₂; and
if R² and R³ are not the same, reacting R¹(NCX)₂ sequentially with R²NH₂ and R³NH₂ in either order.

The various options for X, R¹, R² and R³ described for the first aspect, above, may also apply to the second aspect.

In some embodiments R² and R³ are not the same and the process comprises:
reacting R¹(NCX)₂ with one of R²NH₂ and R³NH₂ in large molar excess of R¹ (NCX)₂;
separating an intermediate adduct from excess R¹(NCX)₂ and;
reacting the intermediate adduct with the other of R²NH₂ and R³NH₂ to produce the compound of structure (A).

In other embodiments R² and R³ are the same and the process comprises reacting R¹(NCX)₂ with about two molar equivalents or slightly more of R²NH₂.

The invention also provides a compound when made by the process of the second aspect.

In a third aspect of the invention there is provided a method for altering the structure of microstructures of an amphiphile in water, said amphiphile being a compound according to the first aspect, said method comprising heating said microstructures in water to a temperature of at least about 60° C., optionally to about 70° C.; and cooling said heated microstructures in water to below about 40° C.

In some embodiments, R³ is a hydrophobic group.

In a fourth aspect of the invention there is provided a method for encapsulating a water soluble substance, said method comprising combining an aqueous solution of said substance with an amphiphile, said amphiphile being a compound according to the first aspect; and sonicating the resulting mixture so as to produce an aqueous product in which at least a portion of the substance is encapsulated within vesicles of the amphiphile.

The following options may be used in conjunction with the fourth aspect, either individually or in any suitable combination.

The method may additionally comprise dialysing the aqueous product so as to remove unencapsulated substance.

The water soluble substance may be a drug.

The vesicles may have a mean diameter of less than about 500 nm, or less than about 400 nm, or less than 300 nm or less than 200 nm. The sonicating may be sufficient (i.e. sufficient time and sufficient power) to generate the vesicles of the desired mean diameter.

R² and R³ in the amphiphile may both be hydrophilic. They may be the same. The amphiphile may be a bolaamphiphile.

In a fifth aspect of the invention there is provided a method for encapsulating a sparingly water-soluble or water insoluble substance, said method comprising combining a solution of said substance with an amphiphile, said amphiphile being a compound according to the first aspect, to form a mixture; and agitating the mixture for sufficient time to form an aqueous product comprising microstructures in which the substance is encapsulated within the amphiphile.

The following options may be used in conjunction with the fifth aspect, either individually or in any suitable combination.

The method may additionally comprise dialysing the aqueous product so as to remove unencapsulated substance.

The substance may be a drug.

R³ may be a hydrophobic group.

The microstructures may have a mean diameter of less than about 500 nm, or less than about 400 nm, or less than 300 nm or less than 200 nm. The agitating may be sufficient (i.e. sufficient time and sufficient power) to generate the vesicles of the desired mean diameter.

In a sixth aspect of the invention there is provided use of a compound according to the first aspect and a drug for the manufacture of a medicament for the treatment of a condition for which the drug is effective.

The condition may be a cancer.

In a seventh aspect of the invention there is provided a method for treatment of a condition, said method comprising administering to the patient a therapeutically effective amount of a drug encapsulated with microstructures, said microstructures comprising an amphiphile which is a compound according to the first aspect and said drug being effective for treatment of said condition.

In an eighth aspect of the invention there is provided use of microstructures comprising a drug encapsulated within an amphiphile in therapy, said amphiphile being a compound according to the first aspect. There is also provided microstructures comprising a drug encapsulated within an amphiphile in therapy, said amphiphile being a compound according to the first aspect. The drug may be indicated for the therapy, i.e. the therapy may comprise treatment of a condition against which the drug is effective.

In a ninth aspect of the invention there is provided a pharmaceutical composition for treatment of a condition, said composition comprising microstructures in which a drug which is effective for treatment of said composition is encapsulated within an amphiphile, said amphiphile being a compound according to the first aspect, said composition additionally comprising one or more pharmaceutically acceptable carriers, diluents and/or adjuvants.

In tenth aspect of the invention there is provided use of an amphiphile which is a compound according to the first aspect for producing microstructures.

The following options may be used in conjunction with the tenth aspect, either individually or in any suitable combination.

The microstructures may encapsulate a substance. The substance may be a drug.

The substance may be sparingly water soluble or water insoluble. In this case and the microstructures may be micelles (optionally swollen micelles) or rod-like structures or emulsion droplets or platelet like structures. R³ may be hydrophobic.

The substance may be water soluble. In this case the microstructures may be vesicles. R³ may be hydrophilic. R² and R³ may be the same.

The microstructures may be less than about 500 nm or less than 400 nm or less than 300 nm or less than 200 nm in mean diameter.

In an eleventh aspect of the invention there is provided a process for producing microstructures, said method comprising dispersing an amphiphile which is a compound according to the first aspect in water. This aspect also encompasses microstructures made by the process.

The following options may be used in conjunction with the eleventh aspect, either individually or in any suitable combination.

The microstructures may encapsulate a substance. In this event, the dispersing may be conducted in the presence of said substance. The substance may be a drug.

The substance may be sparingly water soluble or water insoluble. In this case the microstructures may be micelles or rod-like structures or emulsion droplets or platelet like structures. R³ may be hydrophobic.

The substance may be water soluble. In this case the microstructures may be vesicles. R³ may be hydrophilic. R² and R³ may be the same.

The microstructures may be less than about 500 nm or less than 400 nm or less than 300 nm or less than 200 nm in mean diameter.

In a twelfth aspect of the invention there is provided a method of delivering a substance to a location comprising delivering microstructures (optionally a dispersion of said microstructures in a liquid) to said location, said microstructures comprising the substance encapsulated in an amphiphile according to the first aspect. The delivery may be for a non-therapeutic purpose. It may be for a non-diagnostic purpose. It may be for a non-therapeutic, non-diagnostic purpose. It may be for a therapeutic purpose or for a diagnostic purpose.

### BRIEF DESCRIPTION OF THE DRAWINGS

Preferred embodiment of the present invention will now be described, by way of example only, with reference to the accompanying drawings wherein:

FIG. 1 shows self-assembled morphologies of aqueous solution of amphiphiles 3-5 (about 1 mg/mL). Cryo-TEM (transition electron microscope) image of aqueous solution of 3 showing oligo-lamellar morphology of vesicles (A); TEM image of mixture of spherical and ribbon like morphology of amphiphile 4, immediately after dissolution (B) and after 2 weeks in solution (C); TEM images of rod-like elongated micelles of amphiphile 5 at different magnifications (D and E).

FIG. 2 shows TEM images of 1 wt % aqueous solution of amphiphile 5 with different thermal treatments. Samples were heated at 70° C. for 30 minutes followed by isothermal crystallization: (A) at 4° C. for 12 h; (B) at 22° C. for 12 h; (C) at 37° C. for 12 h; and (D) quenching in liquid nitrogen.

FIG. 3 shows self-assembled morphologies of aqueous solutions of amphiphiles: (A) amphiphile 6, spherical micelles and (B) amphiphile 7, disc-like structures; at about 1 mg/mL.

FIG. 4 is a schematic representation of the process of dox loading into amphiphile 3. Amphiphile 3 was added to a solution of doxorubicin (dox) (A), followed by bath sonication for 5 minutes, resulting in a mixture of free dox and dox loaded amphiphile 3 (B). Free dox was removed by dialysis to result in the final dox-loaded amphiphile 3 (C).

FIG. 5 is a TEM image of 1 wt % aqueous solution of amphiphile 3 loaded with dox (entry 2 in table 4.)

FIG. 6 shows pH dependent cumulative in vitro release profiles of dox from self-assembled amphiphile 3.

FIG. 7 is a graph showing viability of HepG2 cells after being incubated with amphiphile 3, free dox and dox-loaded amphiphile 3 at 37° C. The relative cell viability data suggest that the amphiphile 3 by itself is not toxic to the cells, however the dox-loaded 3 is toxic to the cells, thereby demonstrating that the loaded dox can be released into the cells.

FIG. 8 is a schematic representation of the process of loading dox into a 1 wt % annealed (heated at 70° C. for 30 min, followed by isothermal crystallization at 4° C. for 12 h) aqueous solution of amphiphile 5 (FIG. 8A). Dox and TEA were added to the solution of FIG. 8A and the resultant mixture was allowed to incubate for 12 h (B). Free dox was then removed by dialysis to result in the final dox-loaded amphiphile 5 (C).

FIG. 9 is a TEM image of 1 wt % annealed aqueous solution of amphiphile 5 (heated at 70° C. for 30 minutes, followed by isothermal crystallization at 4° C. for 12 h) and loaded with doxorubicin (entry 2 in Table 4).

FIG. 10 is a graph showing cumulative in vitro release profiles of dox from self-assembled nanostructures. (A) evaluates the of role of thermal treatment on the release of encapsulated dox for amphiphile 5 in PBS (phosphate buffered saline) for unannealed, annealed at room temperature and annealed at 4° C. samples. (B) evaluates the role of pH and composition of the external medium on the release of dox from unannealed amphiphile 5 with phosphate buffers (10 mM, pH 7.4 and 6.5) and PBS (pH 7.4). (C) evaluates the role of pH and composition of the external medium on the release of dox from unannealed amphiphile 6 with phosphate buffers (10 mM, pH 7.4 and 6.5) and PBS (pH 7.4).

FIG. 11 shows results of a cell viability assay of nanostructures loaded with dox, compared with the polymer alone and free dox. (A) amphiphile 5; (B) amphiphile 6.

### DETAILED DESCRIPTION OF THE PREFERRED EMBODIMENTS

The present specification describes design and synthesis of thiourea- and urea-containing amphiphiles for preparation of nanostructures. In particular the design and synthesis of symmetric and asymmetric amphiphiles containing bis-urea or thiourea moieties, and their self-assembly behaviour in water, are described. With the right hydrophobic-hydrophilic balance, the amphiphiles described herein are able to self-assemble in water, for example by direct dissolution, to produce vesicles. These vesicles may be used as carriers for encapsulation and protection of hydrophilic therapeutics and/or nutrients. In other cases, different microstructures may form in water, e.g. micelles, rod-like structures, platelet or disc like structures etc. which may be used as carriers for encapsulation and protection of hydrophobic therapeutics and/or nutrients or other substances.

The general structure of the amphiphiles of the present invention is shown below.

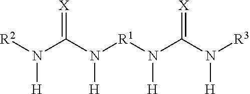

In this structure:

X may be O or it may be S. It is generally the case that both Xs in the structure are the same, i.e. both O or both S, although in some embodiments one of the Xs is S and the other is O. In this case, the X closer to R² may be O or it may be S.
R¹ is a rigid group. In this context, the term “rigid” indicates that it is such that the two nitrogen atoms attached directly to R¹ are maintained in a substantially fixed spatial relationship. R¹ is commonly an aromatic group, or comprises an aromatic group or more than one aromatic group. It may be monocyclic or may be bicyclic or polycyclic. In general the aromatic group will be carbocyclic, however in some instances heterocyclic groups (which may be monocyclic or may be fused ring, optionally fused with a carbocyclic aromatic ring, e.g. benzofurandiyl, benzopyrandiyl etc.) may be used. An aromatic group is a conveniently rigid group and may provide significant hydrophobicity to the amphiphile. Suitable aromatic groups include phenylene (i.e. benzenediyl), which may be ortho or meta or para (i.e. 1,2- or 1,3- or 1,4-), naphthalenediyl, anthracenediyl, phenanthrenediyl, fluorenediyl, acenaphthalenediyl, pyrenediyl, fullerenediyl etc. In some cases R¹ comprises more than one linked aromatic rings/ring systems. For example it may be methylenediphenyldiyl (4,4′, 3,3′, 4,3′ etc.), methylenedinaphthyenediyl, methylenedibenzyl, biphenyldiyl (e.g. 4,4′, 3,3′, 4,3′ etc.) etc. In some instances the aromatic ring of R¹ may be attached to an aliphatic carbon atom to which the adjacent nitrogen atom is attached. Thus R¹ may be for example toluene-α,4-diyl or xylene-α,α′-diyl. Non-aromatic options for R¹ include methylene, ethenediyl (1,2-cis or trans, or 1,1), ethynediyl, conjugated di-, tri- or oligoalkynediyls, aliphatic cage structure hydrocarbons such as norbornanediyl, adamantanediyl, bicyclo[2.2.2]octanediyl and unsaturated analogues thereof etc. Any or all of the above may be optionally substituted for example with one or more alkyl or aryl groups.
R² is a hydrophilic group. It may be non-ionic. It may be non-aromatic. It may have no primary amine groups. It may have no primary or secondary amine groups. It may comprise one or more tertiary amine groups. It may be monomeric, or it may be oligomeric or polymeric. In the present specification, “oligomeric” and related terms encompasses from 2 to 10 monomer units, and “polymeric” and related terms encompasses more than 10 monomer units. R² may be linear or it may be branched. It may comprise one or more ether groups. It may be a polyether. It may be an oligomeric polyether or a polymeric polyether. It may be a non-aromatic oligomeric or polymeric polyether. It may be a branched oligomeric or polymeric polyether or a linear oligomeric or polymeric polyether. It may have oxyethylene monomer units. It may be a linear or branched polyoxyethylene (either oligomeric or polymeric). It may have from about 2 to about 100 ether (e.g. oxyethylene) units, or about 2 to 50, 2 to 20, 2 to 10, 5 to 100, 10 to 100, 50 to 100, 5 to 50, 5 to 20 or 20 to 50 ether units, e.g. about 2, 3, 4, 5, 6, 7, 8, 9, 10, 15, 20, 25, 30, 35, 40, 45, 50, 60, 70, 80, 90 or 100 ether units. R² may have a terminal hydroxyl group or it may have a terminal alkyl (e.g. methyl or ethyl) group or may have some other terminal group. In cases where the number of monomeric units in R² is greater than about 3, it may be inconvenient or difficult to obtain a precursor amine which contains only a single molecular species (i.e. in which each molecule has precisely the same number of monomer units). This difficulty/inconvenience generally increases with increasing numbers of monomer units. In such cases, the stated numbers of monomer units above may represent a mean number of monomer units, and a distribution of numbers, i.e. of chain lengths, may be present. R² may be such that compound (A) is capable of self assembly in water. It may be such that the compound (A) forms vesicles in water, or micelles in water, or emulsion droplets in water, or rod-like aggregates in water, or platelet-like aggregates in water, or some other desired structure. It will be recognised that X, R¹, R² and R³ may all influence the ability of the compound to self assemble in water and may also influence the nature of the aggregates formed in water. For a particular set of X, R¹ and R³, R² may be adjusted in order to achieve the ability to self assemble, and optionally to obtain a desired structure of self assembled aggregates formed in water. Alternatively, R³ may be adjusted for a particular set of X, R¹ and R² so as to achieve these goals. Adjustment of R¹ for a particular set of X, R² and R³ may also influence the ability to self assemble and the structure of self assembled aggregates formed in water. Commonly in order for compound (A) to self assemble in water, R² should be sufficiently large. It may have more than 3 non-hydrogen atoms (or C, N or O atoms) or may have more than 4, 5, 6, 7, 8 or 9 non-hydrogen atoms (or C, N or O atoms). It may have about 4 to about 100 such atoms, or 4 to 50, 4 to 20, 4 to 10, 6 to 50, 6 to 20, 6 to 10, 10 to 100, 20 to 100, 50 to 100, 10 to 50, 10 to 20, 20 to 50 or 2 to 40, e.g. about 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 such atoms.
R³ is a group which may be the same as R² or may be different. In the event that R³ is different to R² it may be hydrophilic or it may be hydrophobic. In the event that it is hydrophilic, it may be as described above for R². In the event that it is hydrophobic, it may be a hydrocarbon group. It may be alkyl or it may be alkenyl or it may be alkynyl. It may be straight chain or it may be branched. It may be, or may comprise, one or more cycloalkyl structures. It may have from about 5 to about 50 carbon atoms, or about 5 to 30, 5 to 20, 5 to 10, 10 to 50, 20 to 50, 10 to 30 or 10 to 20 carbon atoms, e.g. about 5, 6, 7, 8, 9, 10, 11, 12, 13, 14, 15, 16, 17, 18, 19, 20, 25, 30, 35, 40, 45 or 50 carbon atoms. It may have a terminal methyl group or it may have a terminal hydroxyl group or it may have some other terminal group. In the event that it contains one or more carbon-carbon double bonds, each may, independently, be either cis or trans. In some instances R³ may comprise an aromatic group, e.g. a phenyl group, or a heteroaromatic group. In other instances R³ has no aromatic groups. R³ may be optionally substituted, e.g. with one or more alkyl groups, aryl groups, heteroaryl groups or other groups (e.g. functional groups such as hydroxyl). Commonly in order for compound (A) to self assemble in water, R³ should be sufficiently large. It may have more than 3 non-hydrogen atoms (or C, N, S or O atoms) or may have more than 4, 5, 6, 7, 8 or 9 non-hydrogen atoms (or C, N, S or O atoms). It may have about 4 to about 100 such atoms, or 4 to 50, 4 to 20, 4 to 10, 6 to 50, 6 to 20, 6 to 10, 10 to 100, 20 to 100, 50 to 100, 10 to 50, 10 to 20, 20 to 50 or 2 to 40, e.g. about 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 such atoms.

R² may be such that compound (A) can self assemble in water. R² and R³ may be such that compound (A) can self assemble in water. R¹, R² and R³ may be such that compound (A) can self assemble in water.

The HLB (hydrophilic-lipophilic balance) of the compound (A) may be tailored by suitable choice of X and R¹, R² and R³. The HLB may be from about 1 to about 20, or about 1 to 10, 1 to 5, 1 to 2, 2 to 20, 5 to 20, 10 to 20, 2 to 18, 3 to 15, 5 to 10 or 10 to 15, e.g. about 1, 2, 3, 4, 5, 6, 7, 8, 9, 10, 11, 12, 13, 14, 15, 16, 17, 18, 19 or 20. The weight fraction of hydrophobic components in the compound (A) may be less than about 60%, or less than about 50, 40, 30 or 20%, or may be about 10 to about 60%, or about 10 to 50, 10 to 40, 10 to 30, 10 to 20, 20 to 60, 30 to 60 or 20 to 50%, e.g. about 10, 15, 20, 25, 30, 35, 40, 45, 50, 55 or 60%.

The compound (A) may have critical aggregation concentration in water of below about 100 μM, or of less than about 50, 50, 30, 20 or 10, or of about 1 to about 50, or about 1 to 20, 1 to 10, 1 to 5, 5 to 50, 10 to 50, 20 to 50, 5 to 20, 5 to 10 or 10 to 20 μM, e.g. about 1, 2, 3, 4, 5, 6, 7, 8, 9, 10, 15, 20, 25, 30, 35, 40, 45 or 50 μM. It may form micelles in water, or it may form vesicles or it may form laminar structures or it may form rod-like structures or cylindrical structures or platelet-like or disc-like structures in water.

In one particular embodiment of the invention, the amphiphile is:

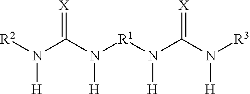

wherein X is either O or S; R1 is a rigid molecule, oligomer or polymer, preferably comprising at least 1 phenyl group; R² is an non-ionic oligomeric or polymeric moiety, excluding amine containing groups; R³ is an non-ionic oligomeric or polymeric moiety, excluding amine containing groups; and wherein at least one of R² and R³ is a hydrophilic moiety.

In another particular embodiment, in the above structure X is either O or S; R¹ is a rigid molecule, oligomer or polymer, preferably comprising at least one phenyl group; R² is an non-ionic oligomeric or polymeric moiety, excluding primary and secondary amine containing groups; R³ is an non-ionic oligomeric or polymeric moiety, excluding primary and secondary amine containing groups; and wherein at least one of R² and R³ is a hydrophilic moiety.

One advantage of the compounds of the present invention is that they may be readily made from commonly available starting materials. They may be made from bisisocyanates (in the event that X is O) or bisisothiocyanates (in the event that X is S). Commonly available bisisocyanates include toluene diisocyanate, methylene diphenyl 4,4′-diisocyanate and phenylene diisocyanate, e.g. 1,4-phenylene diisocyanate. Commonly available bisisothiocyanates include phenylene diisothiocyanate e.g. 1,4-phenylene diisothiocyanate. If necessary, desired bisisothiocyanates and bisisocyanates may be made by commonly known procedures, e.g. by reaction of the corresponding diamines with thiophosgene or phosgene respectively.

In order to produce symmetrical amphiphiles, a bisisothiocyanate or bisisocyanate is reacted with an amine. Commonly the amine is used in approximately two mole equivalents relative to the bisisothiocyanate or bisisocyanate, so that the ratio of amine groups to isocyanate or isothicyanate groups is about 1:1 (since each bisisothiocyanate or bisisocyanate contains two isocyanate or isothicyanate groups). In the event that the amine is expensive or difficult to obtain, it may be used in less than two mole equivalents (e.g. about 1.9, 1.8, 1.7, 1.6 or 1.5 mole equivalents) relative to the bisisothiocyanate or bisisocyanate so as to promote high yield of the reaction with respect to the amine. The molar excess of amine over bisisothiocyanate or bisisocyanate may be about 50 to 200% (i.e. a molar ratio of amine to bisisothiocyanate or bisisocyanate of about 1.5:1 to about 3:1, or amine used in about 1.5 to 3 mole equivalents relative to the bisisothiocyanate or bisisocyanate), or about 50 to 150, 50 to 100, 100 to 200, 100 to 150, 80 to 120, 90 to 110, 100 to 120 or 100 to 110, e.g. about 50, 60, 70, 80, 90, 95, 100, 105, 110, 120, 130, 140, 150, 160, 170, 180, 190 or 200%. The reaction is commonly conducted in a solvent that is capable of dissolving the reagents but not the product. This may be a polar solvent such as methylene chloride. Other suitable solvents include chloroform, THF, diethyl ether etc. The product may be isolated by removal of the solvent, filtration, HPLC, column chromatography, flash chromatography or other suitable method, or by a combination of these. The product may be further purified by washing with a solvent which dissolves the amine but not the product. This may be the same as the reaction solvent or may be different. The reaction may be conducted at or around room temperature (e.g. about 15 to about 30° C.) or at some other convenient temperature and is not particularly sensitive to oxygen or to minor amounts of adventitious moisture. It is therefore a very easy reaction to perform. Yields are commonly high, for example at least about 70%, or at least about 80, 90 or 95%, based on amine or on bisisocyanate or bisisothicyanate. In order to obtain maximum yield a reaction time of at least 30 minutes should be used, or at least about 1, 2, 3, 6, or 12 hours. The reaction time will depend on the reaction temperature, as well as on the nature of the reagents and possibly also on the nature of the solvent.

In order to produce an asymmetric amphiphile the bisisothiocyanate or bisisocyanate is reacted sequentially with two different amines. In the first step the bisisothiocyanate or bisisocyanate is reacted with a first amine (which may have either a hydrophilic or a hydrophobic group attached to the amine nitrogen). As this reaction is intended to produce a monoadduct intermediate (i.e. a 1:1 adduct having a remaining isocyanate or isothiocyanate group), it is common to use a high ratio of bisisothiocyanate or bisisocyanate to amine. Commonly a molar ratio of about 2 to about 10 of bisisothiocyanate or bisisocyanate to amine (corresponding to a ratio of about 4 to about 20 of isocyanate or isothiocyanate groups to amine groups) is used. The molar ratio may be about 2 to 5, 5 to 10 or 3 to 7, e.g. about 2, 3, 4, 5, 6, 7, 8, 9 or 10. In order to prevent a localised high ratio of amine groups to isocyanate or isothiocyanate groups, it is common to add the amine to a solution of the bisisothiocyanate or bisisocyanate, commonly with stirring or other agitation of the solution. This addition may be slow, optionally dropwise. Similar reaction conditions may be used for this reaction (solvent, time, tolerance to oxygen, moisture etc.) as for the symmetric double addition reaction described above. In order to isolate the product, one method is to simply remove solvent. Excess reagents may be removed by washing with a suitable solvent (such as those described above for the symmetric double reaction described above), and further purification of the intermediate may also be similar to that described earlier. The intermediate adduct produced in this way from R¹(NCX)₂ and R^{a}NH₇ (where a is either 2 or 3) is R^{a}NHC(═X)NHR¹NCX. Reaction of this intermediate adduct with R^{b}NH₂ (where, if a is 2 then b is 3 and if a is 3 then b is 2) produces the compound (A). The molar ratio of the intermediate to R^{b}NH₂ is commonly about 1:1, but may be from about 0.8:1 to about 1.2:1 or about 0.8:1 to 1:1, 1:1 to 1.2:1 or 0.9:1 to 1.1:1, e.g. about 0.8:1, 0.9:1, 1:1, 1.1:1 or 1.2:1. Otherwise, reaction conditions and product isolation may be similar to the symmetrical reaction described above.

The compounds of the present invention may be used to form microstructures. Since the compounds can self assemble in water, this may simply be achieved by dispersing the compound in water. The compounds may spontaneously self assemble so as to form the microstructures. The formation of the microstructures may be facilitated by agitation of a mixture of the compound with water. The agitation may comprise shaking, stirring, sonicating or some other form of agitation, or any suitable combination of two or more of these. The agitation may be for sufficient time and with sufficient power/energy to form the microstructures. The sufficient time may depend on the nature of the compound, the concentration of the compound in water, the temperature at which the process is conducted etc. The morphology of the microstructures may also depend on one or more of these factors. The suitable time may be about 1 minute to about 1 hour, or about 1 to 30, 1 to 15, 1 to 10, 1 to 5, 5 to 60, 10 to 60, 30 to 60, 5 to 30, 10 to 30 or 5 to 10 minutes, e.g. about 1, 2, 3, 4, 5, 6, 7, 8, 9, 10, 15, 20, 25, 30, 35, 40, 45, 50, 55 or 60 minutes or may be more than 60 minutes. The temperature may be about 10 to about 50° C., or about 10 to 30, 10 to 20, 20 to 50, 30 to 50 or 20 to 30° C., or about 10, 15, 20, 25, 30, 35, 40, 45 or 50° C., or may be more than 50° C. The microstructures may be micelles, rod-like structures, platelet like structures, vesicles or some other structure. The ratio of compound to water may be about 0.1 to about 50% w/v, or about 0.1 to 20, 0.1 to 10, 0.1 to 5, 0.1 to 1, 1 to 50, 5 to 50, 10 to 50, 20 to 50, 1 to 20, 1 to 10, 1 to 5, 5 to 20 or 5 to 10%. e.g. about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45 or 50% w/v. In some instances the water may contain one or more dissolved substances. It may for example comprise a dissolved salt.

The microstructures may be used to encapsulate a substance. The substance may be water soluble or it may be water insoluble, or it may be sparingly soluble in water. For the purpose of this specification “soluble” indicates a saturation solubility at 20° C. of over about 1% w/v, “sparingly soluble” indicates a saturation solubility at 20° C. of about 0.1 to about 1% w/v and “insoluble” indicates a saturation solubility at 20° C. of less than about 0.1% w/v.

In the event that the substance is water soluble, the encapsulation may be within vesicles. In the context of the present specification, a vesicle is an approximately spherical microstructure present in an aqueous fluid and which encloses an aqueous environment. The walls of a vesicle may comprise a bilayer of amphiphiles, or may, particularly in the case of a bola-amphiphile (which has two or more hydrophilic arms), comprise a monolayer of the amphiphiles in which one arm extends outwards from the vesicle to the aqueous fluid in which the vesicles are present and another arm extends inwards to the enclosed aqueous environment. In some instances vesicles may have multilayer walls, e.g. 2, 3, 4, 5 or more than 5 layers of bolaamphiphile or of amphiphile bilayer. Encapsulation of a water soluble substance may therefore be accomplished by forming vesicular microstructures (as described above) in the presence of the substance. As the vesicles form, the aqueous fluid encapsulated by the vesicle contains the substance which thereby becomes trapped or encapsulated within the vesicles. Any of the substance which is not encapsulated may be removed from the resulting dispersion of vesicles by dialysis, whereby the unencapsulated substance passes through the dialysis membrane and vesicles (containing encapsulated substance) are retained by the membrane. Compounds according to the present invention that are particularly suitable for producing vesicles, and consequently for encapsulating water soluble substances, include compounds in which R² and R³ are both hydrophilic. They may both be oligoether or polyether chains (e.g. oligooxyethylene or polyoxyethylene chains) or may be different hydrophilic groups. These may be regarded as bolaamphiphiles. The walls of vesicles made with these compounds may be monolayers, or may have more than 1 (e.g. 2, 3, 4 or 5) layers.

In the event that the substance is sparingly soluble or water insoluble, it may be encapsulated in a more hydrophobic environment for example the hydrophobic interior of a micelle or of a bilayer or of a rod like structure or of a disc/platelet like structure. In order to accommodate molecules of the substance within these microstructures, it is preferable that the compound (amphiphile) has a hydrophilic chain and a hydrophobic chain, i.e. that R³ is hydrophobic (R² being hydrophilic). The process for encapsulating sparingly soluble or water insoluble substances in these microstructures is similar to that described above for water soluble substances: the microstructures are formed in the presence of the substance. Alternatively, the substance may be added to a preformed dispersion of the microstructures and allowed to diffuse into the microstructures over time. It may be convenient to provide the substance in a solution, or the substance may be provided in a dilute aqueous solution (i.e. sufficiently dilute for the substance to be in solution).

Suitable substances for encapsulation may be drugs (i.e. compounds with biological activity) or visualising substances for imaging purposes. The substance may be an anticancer drug such as doxorubicin. It may be radioisotope labelled substance, e.g. for radiotherapy or for imaging or for both. The substance may be for example a DNA, siRNA, protein, or peptide. It may be a small molecule (e.g. MW less than about 1000) or may be a macromolecule. In some embodiments the substance is a compound that is not biologically active.

In the event that the substance is biologically active, the resulting microstructures may be formulated into a pharmaceutical composition by combination with one or more pharmaceutically acceptable carriers, diluents and/or adjuvants. Suitable carriers, diluents and adjuvants are well known. The composition is thereby suitable for treatment of a condition against which the substance is effective.

The microstructures may have a mean diameter, or a mean hydrodynamic diameter or a mean maximum dimension, of less than about 500 nm or less than 400 nm or less than 300 nm or less than 200 nm, or less than 150, 100, 50 or 20 nm, or of about 10 to about 200 nm, or about 10 to 300, 10 to 400, 10 to 500, 200 to 500, 200 to 400, 100 to 300, 50 to 300, 10 to 100, 10 to 50, 50 to 200, 100 to 200, 50 to 150, 50 to 100 or 100 to 150 nm, e.g. about 10, 20, 30, 40, 50, 60, 70, 80, 90, 100, 110, 120, 130, 140, 150, 160, 170, 180, 190, to 200, 250, 300, 350, 400, 450 or 500 nm. They may have a broad size distribution or a narrow size distribution. They may have a polydispersity index (PDI) of less than about 0.5, or less than about 0.45, 0.4, 0.35 or 0.3, or about 0.1 to about 0.5, or about 0.1 to 0.3, 0.2 to 0.5, 0.3 to 0.5 or 0.2 to 0.4, e.g. about 0.1, 0.15, 0.2, 0.25, 0.3, 0.35, 0.4, 0.45 or 0.5.

In some instances it may be preferable to alter the morphology and or size of the microstructures. This may be done before or after loading them with a substance. This may be accomplished by an annealing process. Thus the microstructures may be heated to a suitable high temperature, optionally maintained at that temperature for a suitable time, and then cooled to a suitable low temperature. The suitable high temperature may be over about 60° C., or over about 70 or 75° C., or may be about 60 to about 100° C., or about 60 to 90, 60 to 80, 70 to 100, 70 to 90 or 70 to 80° C., e.g. about 60, 65, 70, 75, 80, 85, 90, 95 or 100° C. The suitable time may be at least about 5 minutes, or at least about 10, 15, 20, 25 or 30 minutes, or about 5 to about 60 minutes, or about 5 to 30, 5 to 20, 5 to 10, 10 to 60, to 60, 30 to 60 or 20 to 40 minutes, e.g. about 10, 15, 20, 25, 30, 35, 40, 45, 50, 55 or 60 minutes. The low temperature may be less than about 40° C., or less than about 30, 20, 10 or 0° C., or about 0 to 40, 0 to 30, 0 to 20, 0 to 10, 10 to 40, 20 to 40, 30 to 40 or 20 to 30, e.g. about 0, 5, 10, 15, 20, 25, 30, 35 or 40° C., or may be below 0° C., e.g. liquid nitrogen temperature or −78° C. The low temperature may be maintained for sufficient time to form the desired morphology of the microstructures. It may be maintained for example for at least about 5 minutes, or at least about 10, 15, 20, 25 or 30 minutes, or about 5 to about 60 minutes, or about 5 to 30, 5 to 20, 5 to 10, 10 to 60, 20 to 60, 30 to 60 or 20 to 40 minutes, e.g. about 5, 10, 15, 20, 25, 30, 35, 40, 45, 50, 55 or 60 minutes. This alteration may be particularly suitable to microstructures comprising asymmetric compounds (A), e.g. those in which R³ is hydrophobic. It may be for the purpose of rendering the microstructures more suitable for biological, e.g. therapeutic or imaging, applications or it may be for the purpose of rendering them more suitable for a non-biological (e.g. non-therapeutic and/or non-imaging) purpose.

Microstructures of the compound (A) may be used to deliver a substance (either hydrophilic or hydrophobic) to a desired location. In some cases the substance is biologically active, or may be radioactive, and the delivery is for therapeutic purposes. For example the substance may be a drug. It may be an anticancer drug, whereby the delivery may be to the site of a tumour for the purposes of treating the tumour. In other cases the substance may be visualisable. It may be radioactive, or it may be a contrast agent or may be some other visualisable substance. In this case the delivery may be for the purpose of diagnosis of a condition in a patient. In the above cases the patient to whom the substance is delivered may be human or may be non-human. The patient may be a non-human primate, or may be a non-human non-primate patient. In yet other examples the desired location to which the substance is delivered is a non-biological location. The delivery may be for a non-therapeutic purpose. It may be for a non-diagnostic purpose. It may be for example for an industrial purpose, e.g. delivery of an enzyme for an in vitro transformation. Release of the substance from the microstructures may comprise changing the environment in which the microstructure is disposed. It may comprise acidifying the microstructures (said microstructures having the substance encapsulated within the compound (A)). It may for example comprise reducing the pH to less than about 5, or less than about 4.5, 4, 3.5 or 3, or to about 2 to about 5, or about 2 to 4, 2 to 3, 3 to 5 or 3 to 5, e.g. to about 2, 2.5, 3, 3.5, 4, 4.5 or 5. It may comprise subjecting the microstructures having the substance encapsulated within the compound (A) to an environment in which the substance of compound (A) degrades so as to cause the microstructures to deteriorate. For example it may comprise exposing the microstructures to a hydrolytic environment.

In this work, the facile synthesis of amphiphiles containing bis-urea or thiourea moieties of structure (A) is described, as well as the self-assembly of these amphiphiles into vesicles.

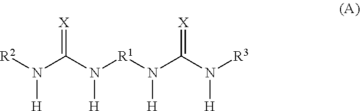

Structure (A) represents a general structure of the class of amphiphiles containing rigid hydrogen bonding thiourea or urea containing molecular recognition units, i.e. X is either S or O. For symmetric amphiphiles, R¹ is a rigid group; R² and R³ are the same, and are a hydrophilic group, optionally oligomeric or polymeric. For asymmetric amphiphiles, R¹ is a rigid group; R² is a hydrophilic molecule, optionally oligomeric or polymeric and R³ is a hydrophilic group or a hydrophobic group, optionally oligomeric or polymeric, and R² and R³ are different.

The bis-urea or thiourea moieties along with R¹ constitute the central hydrophobic molecular recognition part. When R² and R³ are hydrophilic and the same, the compound (A) is a symmetric bolaamphile. When they are different, compound (A) is an asymmetric structure. The inventors hypothesized that with the right hydrophobic-hydrophilic balance (HLB), these compounds would self-assemble in aqueous environment to form well-defined nanostructures. In this specification the synthesis of these compounds is described, along with the evaluation of their self-assembly into nanostructures. Encapsulation of doxorubicin, an anti-cancer drug, into these self-assembled nanostructures has also been explored as a model encapsulated substance. An advantage of this as a model is that it may be relatively hydrophobic in its neutral form, or may be relatively hydrophilic in its protonated form. These forms may simply be interchanged by altering the pH of the solution.

Advantages of the technology of the present invention include:
Vesicles can be formed by direct dissolution in water, containing therapeutics and/or nutrients of particular compounds according to the invention. Therefore, they represent a promising carrier for encapsulation and protection of water soluble therapeutics and/or nutrients;
Synthesis of the compounds is readily scalable.
These compounds have a potential application in the encapsulation and protection of water soluble therapeutics and/or nutrients. Therefore, they may be of great interest to pharmaceuticals and cosmetics industries.

### Synthesis of Symmetric Amphiphiles

For the preparation of symmetrical thiourea containing amphiphiles (1-3), 1,4-bis-phenyl isothiocyanate (BPTC) was used as the precursor for both the hydrophobic and hydrogen-bonding motif. Moreover, the isothiocyanates are selective in their reactivity towards amines in the presence of numerous other functionalities, allowing for the placement of functionalities that can be further manipulated for additional conjugations. Different amino alcohols were reacted with BPTC in the chloroform to result in the amphiphile with central hydrophobic thiourea motif in high yields with simple purification steps.

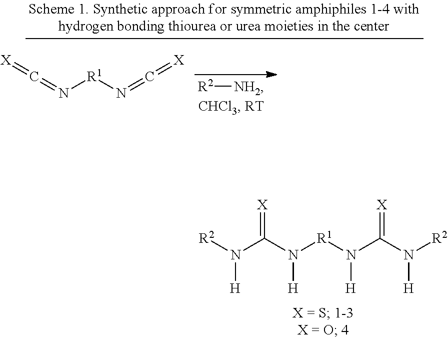

TABLE 1
List of specific amphiphiles containing rigid hydrogen bonding thiourea or urea
components described in this specification.

| S. | | | | |
|---|---|---|---|---|
| No.: | X | R¹ | R²NH₂ | R³NH₂ |
| 1 | S | 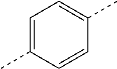 | 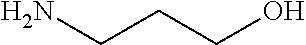 | 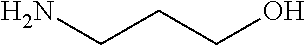 |
| 2 | S | 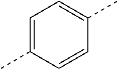 | 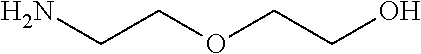 | 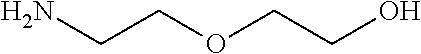 |
| 3 | S | 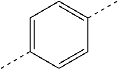 | 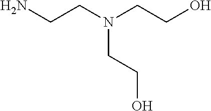 | 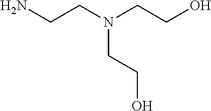 |
| 4 | O | 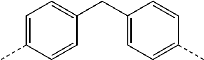 | 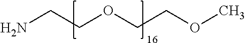 | 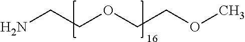 |
| 5 | S | 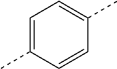 | 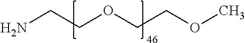 | 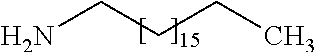 |
| 6 | S | 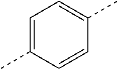 | 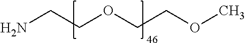 | 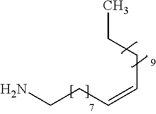 |
| 7 | S | 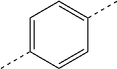 | 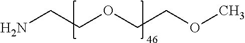 | 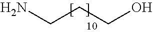 |

Compared to thiourea, the associative interaction of urea moieties are known to be stronger and hence, it would be useful to incorporate bis-urea moiety in an amphiphilic structure. To accomplish this, commercially available methoxy-PEG-NH₂ (750 Da) was reacted with 4,4′-methylene-bis-(phenylisocyanate) to yield 4. Moreover, incorporation of PEG (polyethylene glycol) can improve the stability of the nanostructures especially in a serum-containing medium.

### Synthesis of Asymmetric Amphiphiles

Asymmetric amphiphiles were prepared in a two-step process.

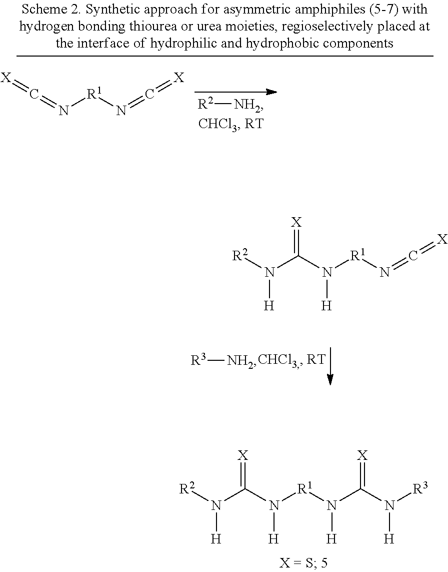

First, octadecyl amine was reacted with tenfold excess of BPTC to result in monosubstituted intermediate. The excess of BPTC was washed away by using the diethylether. The purified monosunstituted intermediate was reacted further with methoxyPEG-NH₂ (˜2000 Da) and the final compound was purified by flash column chromatography to result in pure product 5. This strategy has been extended to other functional amino, alcohols, including oleyl amine and 1-amino dodecanol to result in amphiphile 6 and 7 respectively. The alcohol functionality in 7 can be potentially used as for further chemical manipulation, including as an initiator for subsequent ring opening polymerization.

### Molecular Self-Assembly

### Self-Assembly of Symmetric Amphiphiles 1-4

The self-assembly behavior of the all the amphiphiles in water was evaluated. Compounds 1 and 2 were found to have poor water solubility. This might be due to the fact that the high weight fraction of the hydrophobic thiourea groups. Hence their self-assembly behaviors were not evaluated.

Compound 3, with weight fraction of hydrophilic moieties (0.54) was found to, self-assemble in water (Table 2.).

TABLE 2
Self assembly behavior of amphiphiles in water

| | Weight | Critical | |
|---|---|---|---|
| | fraction of | aggregation | Hydrodynamic |
| S. | hydrophobic | concentration, | diameter, nm |
| No.: | components | (CAC)^{c} | (PDI) |
| 1 | 0.66^{a} | NA | NA |
| 2 | 0.56^{a} | NA | NA |
| 3 | 0.46^{a} | 2.5 ppm | 310 (0.28) |
| | | (5.1 μM) | |
| 4 | 0.16^{a} | 25 ppm | 313 (0.35) |
| | | (14.3 μM) | |
| 5 | 0.19^{b} | 30 ppm | 84 (0.26) |
| | | (13.0 μM) | |
| 6 | 0.19^{b} | NA | NA |
| 7 | 0.17^{b} | NA | NA |

NA. not available.
^{a}only the bis thiourea or urea groups along with the connecting aryl group was used as the hydrophobic part in the calculation. The rest of the molecules were treated as the hydrophilic part.
^{b}the bis thiourea or urea groups along with the connecting aryl group and the alkyl groups of the hydrophobic amine were used as the hydrophobic part in the calculation.
^{c}Based on derived count rates obtained from DLS (dynamic light scattering) measurements of 3 × 10, 1 second runs for each concentration.

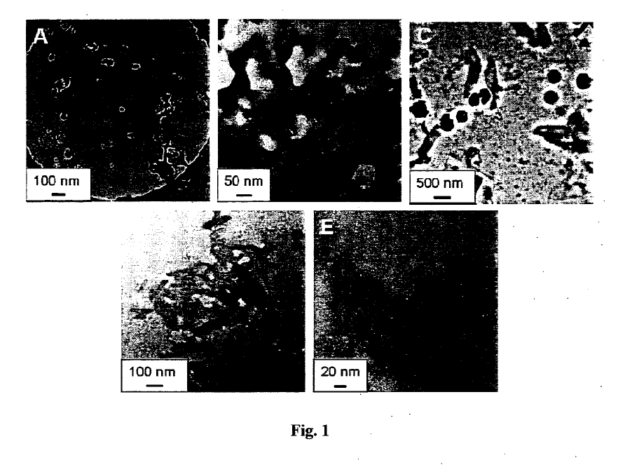

CAC as determined from the intensity counts of serially diluted samples, was found to be low (˜2.5 ppm). The typical size hydrodynamic diameters of the self-assembled particles were about 300 nm. With mild bath sonication (10 min), it was found that the polydispersity of these particles could be brought down to about 0.28. From, cryo-TEM images, they were found to be oligo-lamellar vesicles (FIG. 1 image A.). These vesicles could be potentially applied for the encapsulation of water soluble compounds.

PEGylated symmetrical and asymmetric amphiphiles were also found to readily self-assemble in water to result in nanoparticles (Table 2). Even with higher weight percentage of hydrophilic content, these amphiphiles retained relatively low CAC of about ≦30 ppm, rendering these nanoparticles attractive for biomedical applications. The symmetrical amphiphile 4, at 1 mg/mL concentration, was found to form a mixture of spherical nanostructures (diameter ˜30-60 nm) and twisted and entangled nanoscale ribbons (width ˜15 nm; length—50 nm to 1 μm, (FIG. 1 image B). As both the nanoscale ribbons and spherical nanoparticles no longer appear after 2 weeks, indicating that these structures may not be thermodynamically stable (FIG. 1 image C). After about 2 weeks in solution, the predominant morphology was found to be mixture of vesicles (diameter ˜500 nm) and tubules (diameter ˜500 nm and length ˜1-3 μm).

### Self-Assembly of Asymmetric Amphiphiles 5-7

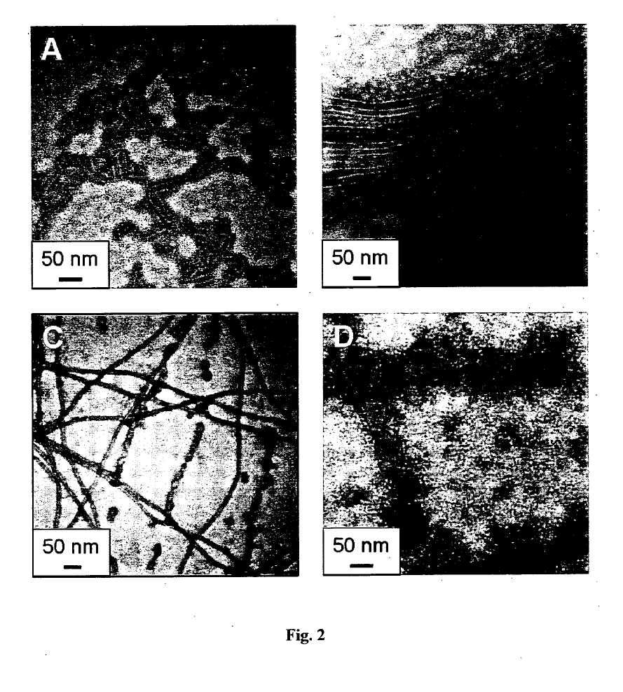

The asymmetric amphiphile 5, at 1 mg/mL concentration, amphiphile was found to form nanostructures with high aspect ratio (diameter about 15 nm; length about 100 nm to 1 μm, FIG. 1 images D and E). The elongated morphology could be due to the introduction of both the C18 crystalline alkyl group and the rigid thiourea motif. For enhanced cellular uptake, it is desirable to have nanostructures of size below about 200 nm. In order to tune the lengths and morphology of these self-assembled nanostructures annealing was explored. An amphiphile 5 solution was subjected to thermal treatment by heating to 70° C. for 30 minutes followed by isothermal crystallization at different temperatures for 12 h (FIG. 2). Upon isothermal crystallization at 4° C., short rods (diameter about 15 nm and length about 20-60 nm) were found to be formed. At 22° C., long rods, extending up to several microns were formed along with some spherical micelles. These elongated structures were found to be aligned along their length as bundles. As the hydrophobic core is annealed, the tethering density of PEG would be expected to increase, leading to such alignment due to the attraction of the PEG shell between the nanostructures. Upon increasing the isothermal crystallization temperature to 37° C., network of elongated micelles along with pearl-necklace like aligned micellar structures were formed. To confirm that these morphological changes are primarily driven by the thermal treatment, the amphiphile solution which had been heated at 70. ° C. for 30 minutes was immersed into a bath of liquid nitrogen and the morphology of self-assembly was investigated. Only spherical micellar morphology (diameter about 40-100 nm) was formed. These results suggest that the morphology of amphiphiles with crystalline components can be tuned by thermal treatment of the samples.

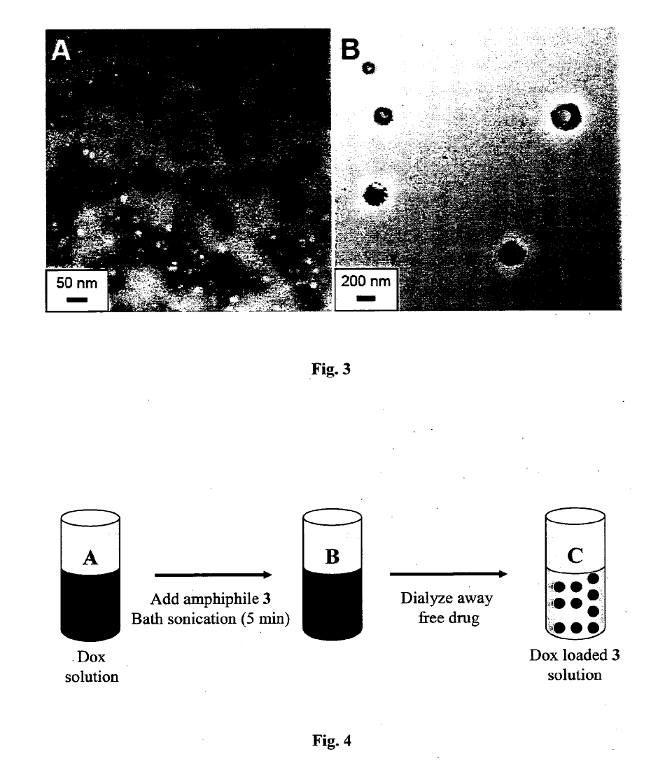

Compared to amphiphile 5, amphiphile 6 has one unsaturated bond, which amounts to a difference of only two hydrogen atoms, yet aqueous self-assembly of 6 result in well defined uniform spherical micelles of about 15 nm in diameter (FIG. 3 image A). It is remarkable to find that such a small difference in mass, with profound changes in physical properties (crystallinity vs. fluidity), but without the direct perturbation of hydrophobic-hydrophilic balance, can indeed affect the self-assembly behaviours. When the hydrophobic part of the asymmetric amphiphile was changed to 1-amino dodecanol as in the case of amphiphile 7, only disc-like morphology was observed (FIG. 3 image B). These findings suggest that controlling the composition of the amphiphiles is indeed a viable strategy to access a variety of nanostructures.

### Evaluation of Amphiphiles as a Potential Carrier for Doxorubicin (Dox)

### Symmetric Amphiphile 3:

In order to investigate the encapsulation of hydrophilic drugs into the vesicles formed by amphiphile 3, dox was used in the form of its hydrochloride salt. To a solution of dox, amphiphile 3 was added and was allowed to self-assemble. Free dox was removed by dialysis (FIG. 4). It was found that the ratio of the amphiphile to drug influenced the size distribution of the drug loaded vesicles: the higher the amphiphile to dox ratio, better the size distribution (Table 3).

TABLE 3
Optimization of dox loading into the vesicles,
self-assembled from amphiphile 3

| S. | 3/Dox | % Loading | Size, D_{z} | |
|---|---|---|---|---|
| No. | (wt. ratio) | efficiency | (nm) | PDI |
| 1 | 4 | 24.8 | 178 | 0.27 |
| 2 | 2 | 21.8 | 156 | 0.32 |
| 3 | 1 | 26.9 | 187 | 0.48 |

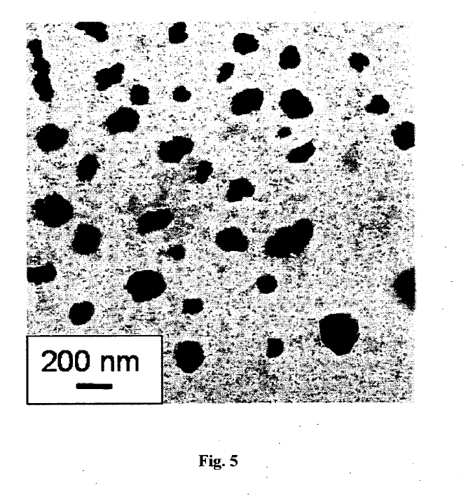

However, the loading efficiency (in the range of about 22-27%) of the dox encapsulated amphiphile, remained comparable. The hydrodynamic diameter as measured by DLS measurements was less than 200 nm for all the conditions. The size of the dox loaded amphiphile 3, as observed from TEM (FIG. 5) is also in agreement with the DLS measurements.

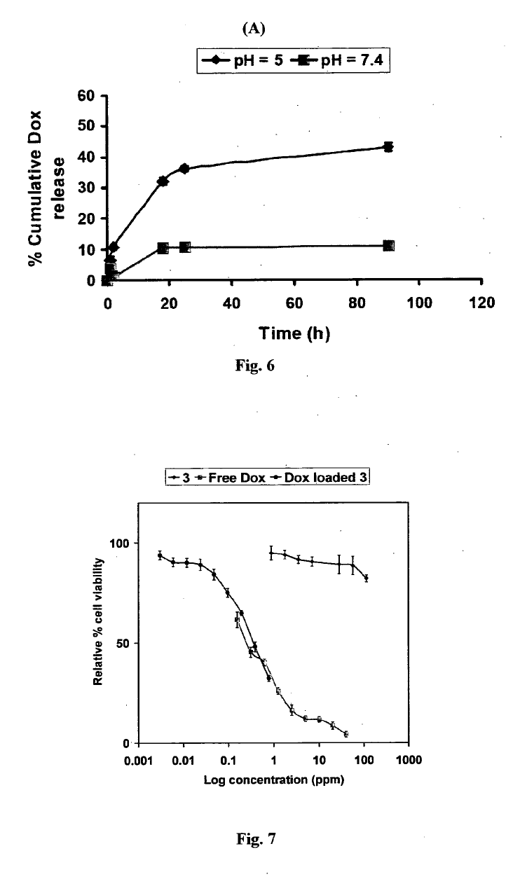

Studies to evaluate the in vitro release of dox demonstrated a pH dependence (FIG. 6). A higher amount of dox was released under acidic conditions (pH=5.0), when compared to neutral conditions (pH=7.4). After 80 hours, about 40% of the loaded dox was released at pH 5.0 while only 10% was release at pH 7.4. FIG. 6 shows this effect for two different amphiphiles (A). The pH dependent release profile offers a simple handle to trigger release of the encapsulate contents. Compared to non-tumorous sites, tumor sites are known to be acidic. Thus this pH dependent release may have implications on the delivery of anticancer therapeutics. To evaluate the bioavailability of the release dox, the inventors conducted cell viability assay against HepG2 cells (FIG. 7). The cytotoxicity profile of the dox encapsulated amphiphile 3, had an IC₅₀ (concentration to achieve 50% cell death) comparable to that of free dox, while the amphiphile alone was not toxic.

### Asymmetric Amphiphiles:

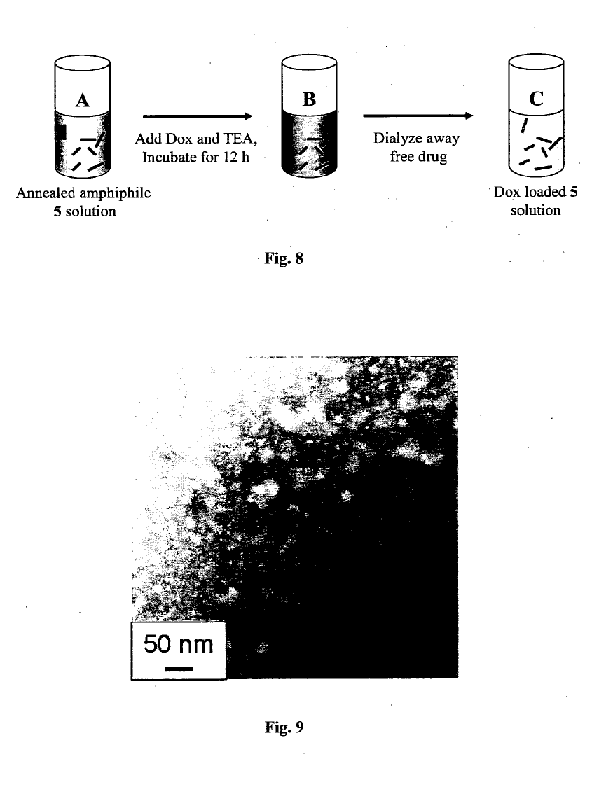

The role of size and shape of nanoparticles for drug delivery applications has been systematically explored previously, and it appears that short rod-like morphologies are promising candidates. Hence for the drug encapsulation related experiments, the inventors focussed on short rods, obtained by annealing at 4° C. (FIG. 2 image A). Dox was found to partition into the hydrophobic core of these rods. The capsulation efficiency was about 19%. To improve the encapsulation efficiency, triethylamine was added (FIG. 8). In the presence of triethylamine, dox is rendered neutral and relatively more hydrophobic that the salt form. In this way the encapsulation efficiency was significantly improved to about 69% (Table 4), without significantly altering the morphology (FIG. 9).

TABLE 4
Optimization of Dox loading into the self-assembled amphiphile 5

| S. | 5/Dox | Triethyamine/Dox | % Loading |
|---|---|---|---|
| No. | (wt. ratio) | (molar ratio) | efficiency |
| 1 | 5 | — | 19.2 |
| 2 | 5 | 3.0 equiv. | 69.0 |

With this optimized protocol, Dox was encapsulated into the nanostructures formed by both 5 and 6. Also, the solutions of 5 with prior thermal treatment were also used to encapsulate dox. Typically dox loading of about 14 wt % with an encapsulation efficiency of up to about 60% was achieved (Table 5).

TABLE 5
Summary of dox-loaded nanostructures prepared from amphiphile 5 and 6

| | | | Hydrodynamic |
|---|---|---|---|
| | Drug loading | | diameter |
| | Loading | Encapsulation | Intensity |
| Sample | (wt.%) | efficiency % | mean |
| 5 (Unannealed) | 14.4 ± 0.9 | 66.3 ± 10.7 | 132 ± 05 |
| 5 (Annealed @ 4° C.) | 14.1 ± 0.3 | 60.3 ± 8.5 | 133 ± 33 |
| 5 (Annealed @ RT) | 14.9 ± 0.5 | 69.6 ± 2.0 | 197 ± 31 |
| 6 | 16.3 ± 2.3 | 58.3 ± 2.1 | 274 ± 50 |

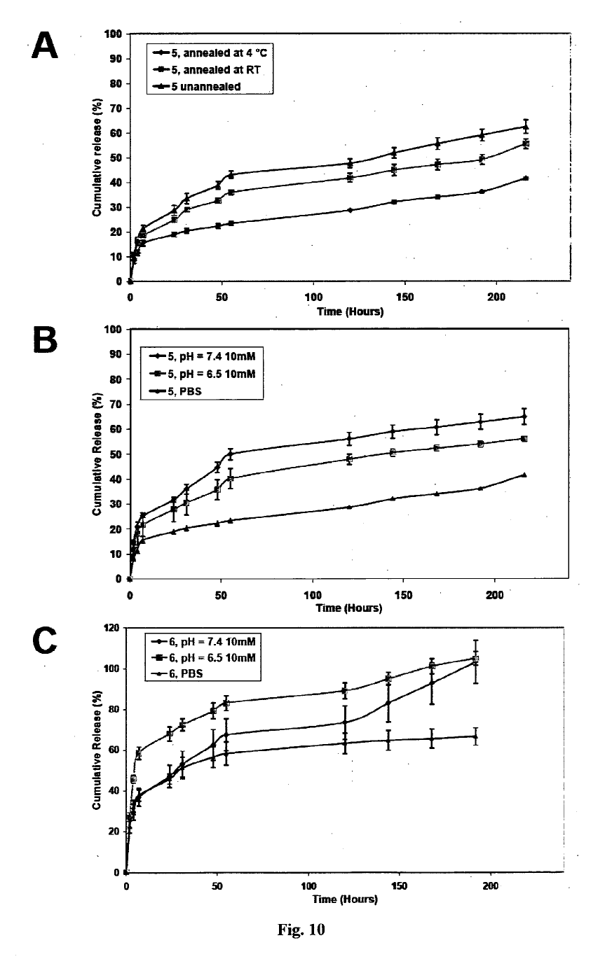

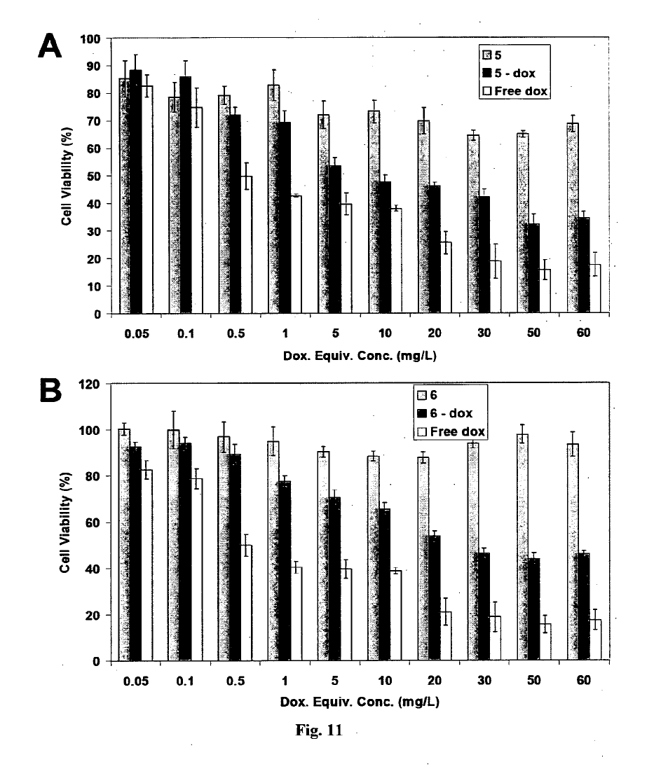

The encapsulated dox was released over time, and the release rates were pH and salt concentration dependent. Release rates were faster in phosphate buffers (10 mM, pH 6.5 or 7.4) than PBS (pH 7.4). Also compared to amphiphile 5, complete release of encapsulated dox was observed over 200 hours with phosphate buffers for the amphiphile 6. However, the release trends of acidic condition (pH=6.5) compared to pH=7.4, were different for the amphiphiles 5 and 6. For the amphiphile 6, in acidic environment faster dox release was observed when compared with pH=7.4. A reverse trend was observed amphiphile 5, along with the general difference in release rates, pointing out that the nature of the hydrophobic component can influence the drug release behaviors (FIGS. 10 graphs B and C). As for the nanostructures prepared with thermal treatments, even though the loading contents were similar, the release rates were significantly different (FIG. 10 graph A). The untreated sample gave rise to the slowest release, followed by the sample in which the isothermal crystallization was allowed to occur at room temperature, and the sample with isothermal crystallization allowed to occur at 4° C. This finding highlights the effect of packing nature in the core on the drug release behaviors. To evaluate the in vitro bioavailability of the released Dox, cell viability assays were conducted against HepG2 cells (FIG. 11 graphs A and B). Dox-loaded amphiphiles 5 and 6 had higher IC₅₀ as compared to free Dox. Amphiphile 5 was slightly cytotoxic at high concentrations, while amphiphile 6 was not cytotoxic even at high concentrations.

### CONCLUSIONS

With commercial starting materials, facile and efficient synthetic routes for symmetric and asymmetric amphiphiles containing rigid hydrogen-bonding bis-urea or thiourea moieties have been developed. With balanced amphiphilicity, these amphiphiles can self-assemble in aqueous environment to yield numerous nanostructures. Both the exact chemical composition and also the nature of packing (due to the thermal treatment) were found to be crucial for the morphological outcomes of self-assembly. A simple and efficient drug encapsulation methodology, eliminating the use of organic solvents has been developed. The release of the encapsulated drug was found to be tunable (by pH, buffer conditions, nature of core packing via thermal treatment). These nanostructures can potentially be used for delivery of hydrophobic and hydrophilic therapeutics, including peptides, proteins and siRNA.

### Experimental Section

### Materials

All reagents were purchased from Sigma Aldrich® or Merck® or Alfa Aesar® and used as received. All other solvents were of analytical grade, purchased from Merck® or J. T. Baker® and used as received.

### Methods

¹H- and ¹³C nuclear magnetic resonance (NMR) spectra were recorded using a Bruker Avance spectrometer (400 and 100 MHz respectively), on solutions with the solvent signal as the internal reference standard. Dynamic Light Scattering (DLS) studies were conducted in Malvern® Nano ZS90 instrument. For CAC measurements, derived count rates from DLS were used. Matrix assisted laser desorption ionization time-of-flight mass spectroscopy (MALDI-TOF) was performed on a Bruker® autoflex II spectrometer using an α-cyano-cinnapinic acid matrix. Transmission electron microscopy (TEM) images were obtained using FEI® Tecnai G² F20 electron microscope using an acceleration voltage of 200 eV. 2 wt. % phosphotungstic acid was used as a staining agent. Typically 8 μl, of sampled is placed on the grid and waited for 1 minute and the excess sample is wicked off. Then 8 μL of the staining solution is placed on the grid and after 1 minute the excess staining solution is wicked off. Finally the grids are dried in ambient conditions. Sample for cryogenic TEM were prepared in a Vitrobot® instrument at room temperature and a relative humidity >95%. A 3 μL of sample was applied to a Quantifoil® grid (freshly glow discharged just prior to use), the surplus solution were blotted away and the grid containing thin film was shot in liquid hydrochlorofluorocarbon (HCFC) based refrigerant. The vitrified film was transferred to a cryo-holder and observed using FEI® Tecnai G² F20 electron microscope.

General Procedure for Synthesis of Symmetric Amphiphiles (1-3) with Thiourea Motifs:

To a 0.5 M solution of BPTU (1,4-diisothiocyanatobenzene) in CHCl₃, 2.05-2.1 equivalence of amino alcohol was added. Precipitates of the bis-thiourea product were observable within 30 minutes. The reaction mixture was allowed to stir over night and the product was isolated by filtration. The product was washed with about 10-20 mL of solvent to remove the excess amino alcohol.

1: ¹H NMR (400 MHz, DMSO-D₆, δ, ppm): 7.30 (s, 4H, Phenyl), 3.6-3.4 (m, 8H, CH₂CH₂CH₂OH), 1.68 (qn, 4H, CH₂CH₂CH₂OH). ¹³C NMR (100 MHz, DMSO-D₆, δ, ppm): 180.9, 135.9, 124.2, 59.4, 42.2, 32.3.

2: ¹H NMR (400 MHz, DMSO-D₆, δ, ppm): 7.35 (s, 4H, Phenyl), 3.7-3.4 (m, 16H, CH₂O), 1.68 ¹³C NMR (100 MHz, DMSO-D₆, δ, ppm): 180.8, 135.7, 123.7, 72.5, 69.0, 60.7, 44.0.

3: ¹H NMR (400 MHz, DMSO-D₆, δ, ppm): 7.3 (s, 4H, Phenyl), 3.7-3.3 (m, 12H, NHCH₂ and CH₂OH), 2.8-2.5 (m, 12H, NCH₂) ¹³C NMR (100 MHz, DMSO-D₆, δ, ppm): 180.7, 136.0, 124.1, 59.7, 57.4, 57.1, 53.7, 42.6.

### Synthesis of 4:

To a solution of 4,4′-methylene-bis-(phenylisocyanate) (35 mg) in CHCl₃ (3.5 mL), methoxy-PEG-NH₂ (206 mg, corresponding to about 2 equivalents with respect to the —NCO groups) was added. The reaction was allowed to stir under argon for overnight. TLC (CH₃OH:CHCl₃=1:9), indicated that the reaction went to completion. Solvent was removed under vacuum and the final product was obtained as a yellowish solid. ¹H NMR (400 MHz, CHCl₃-D, δ, ppm): 7.3 (d, 4H, Phenyl), 7.05 (d, 4H, Phenyl), 3.8-3.4 (m, 136H, CH₂O), 3.35 (s, 3H, OCH₃). MALDI-TOF [M+Na⁺]=1830.8

### General Procedure for Synthesis of Asymmetric Amphiphiles.

### Synthesis of 5:

Octadecyl amine was reacted with tenfold (with respect to —NCS group) excess of BPTC in CHCl₃ to result in monosubstituted intermediate. The intermediate compound had poor solubility in CHCl₃ and appeared as milky suspension in CHCl₃. Solvent was removed and the resultant white solid was washed extensively with diethylether to remove the unreacted BPTC starting materials. The purified monosubstituted intermediate was reacted further with methoxy-PEG-NH₂ (˜1900 Da, Sun Bio) in CHCl₃. The final compound was purified by flash column chromatography with solvent gradient from pure CHCl₃ to CH₃OH:CHCl₃=1:9 to yield final product as white solid.

¹H NMR (400 MHz, CHCl₃-D, δ, ppm): 7.6-7.3 (m, 4H, Phenyl), 7.05 (d, 4H, Phenyl), 3.8-3.4 (m, 170H, CH₂O and NHCH₂), 3.35 (s, 3H, OCH₃), 1.7-1.2 (m, 32H, CH₂), 0.88 (t, 3H, CH₃). MALDI-TOF [M+Na⁺]=2292.8

### General Procedure for Loading of Doxorubicin (Dog) in Amphiphile 3:

To a 10 mL of aqueous dox.HCl solution (concentration=0.5 mg/mL, entry 2, Table 3), 10 mg of 3 was added. The mixture was bath sonicated for 15 min with gentle heating (about 37-40° C.). The reaction mixture was equilibrated for at least 2 hour at room temperature with stirring and then transferred to a dialysis bag with a molecular cut off of 1 kDa and dialyzed for 18 hours in a beaker 800 ml deionised water. The water was changed in the 2^{nd}, 6^{th} and 12^{th} hours. Dox loading was determined by measuring absorbance at 480 nm by UV-Vis spectrophotometer after diluting 100 μL of the solution 10 times with 9:1 DMSO:H₂O with 1.5 wt % TFA. Dox concentration was calculated by comparing the absorbance with dox calibration curve in 9:1 DMSO:H₂O with 1.5 wt % TFA (trifluoroacetic acid).

### General Procedure for Loading of Dox in Asymmetric Amphiphiles:

Drug Loading with Annealed Polymer

About 5 mg of 5 was dissolved in 2.5 mL of DI water by bath sonication for 10 minutes to obtain a 2 mg/mL solution. The resulting solution was heated at 70° C. for 1 hour and annealed at either 4° C. or room temperature for 7 hours. The annealed solution was then added to 1 mg DOX and 2.5 μL TEA (1:3 molar ratio). 2.5 ml of DI water was then added and the mixture was stirred for 15 hours. The reaction mixture was then transferred to a dialysis bag with a molecular cut-off of 1 kDa and dialyzed for 24 hours with 1 L of DI water. The water was changed at 2^{nd}, 4^{th} and 7^{th} hours. Dox loading was determined by measuring absorbance at 480 nm with a UV-Vis spectrophotometer after freeze-drying.

Drug Loading with Unannealed Polymer

About 5 mg of amphiphile 5 dissolved in 2.5 mL of DI water by bath sonication for 10 minutes. In a separate tube, 1 mg dox and 2.5 μL TEA (triaethylamine; 1:3 molar ratio) was dissolved in DI water by intense vortexing for 20 min. The solution of amphiphile 5 was then added to the dox mixture and stirred for 15 hours. The reaction mixture was then transferred to a dialysis bag with a molecular cut-off of 1 kDa and dialyzed for 24 hours with 1 L of DI water. The water was changed at 2^{nd}, 4^{th} and 7^{th} hours. Dox loading was determined by measuring absorbance at 480 nm by UV-Vis spectrophotometer after freeze-drying.

### In Vitro Release:

The in vitro release of dox was studied in fresh PBS buffer (150 mM, pH 7.4) and phosphate buffer (10 mM, pH 6.5 and pH 7.4). Dox-loaded amphiphile solutions (2 mL) were transferred to three dialysis bags with molecular weight cut-off of 2 kDa. The dialysis bags were rinsed with DI water and soaked in filter paper to remove any leached Dox solution before immersing them into 20 mL PBS or phosphate buffers respectively in plastic tubes. The tubes were shaken at 100 rpm and incubated at 37° C. for 216 hours. About 1 mL of buffer solution was withdrawn and replaced with the respective buffer at specific time points. The released dox concentrations in the 1 mL solutions were measured by a UV-Vis spectrophotometer at 480 nm. A graph of cumulative percentage of dox released was plotted against time.

### Cell Viability Assay:

Cell viability of the free amphiphiles, amphiphiles loaded with dox and free dox were examined at various concentrations by MTT (3-(4,5-dimethylthiazol-2-yl)-2,5-diphenyl tetrazolium bromide) assay. HepG2 cells were seeded in 96-well micro-plates at a cell density of 8000 cells per well and cultivated in 100 μL DMEM medium. They were incubated at 37° C. and supplied with 5% CO₂ for 24 h, after which the spent medium was replaced with 100 μL of new medium and 10 μL of solutions of amphiphile, amphiphile loaded with dox and free dox were examined at various concentrations. Each concentration was prepared sufficient for 8 replications and the negative control used was treated with the respective buffer. The micro-plates were left in the incubator for a period of 4% before they were replaced with fresh medium and returned to the incubator for another 48 h. New medium (100 μL) containing 10 μL of MTT solution at 5 mg/ml were used to replace the spent medium in each well and the plates were left in the incubator. After 4 h, the DMEM and excess MTT in each well were removed and 150 μL of DMSO (J.T Baker) was added to each well to dissolve the purple formazan crystals. The micro-plates were left to mix thoroughly on a plate shaker for 5-10 min. 100 μL of the homogenized sample from each well was transferred to a new plate and the absorbance rate of each sample was read at 550 nm and 690 nm with a micro-plate reader (PowerWave® X, Bio-Tek Instruments®). The results from the readings were expressed as a percentage of the negative control.

1. A compound of structure (A) 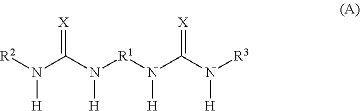
wherein:
X is either O or S;
R¹ is a rigid group;
R² is a hydrophilic group such that (A) is capable of self-assembly in water; and
R³ is an organic group.

2. The compound of claim 1 wherein X is S.

3. The compound of claim 1 wherein at least one of R² and R³ is oligomeric or polymeric.

4. The compound of claim 1 wherein R² comprises an oligoether or a polyether chain.

5. The compound of claim 4 wherein the oligoether or polyether chain is an oligo- or poly-oxyethylene chain.

6. The compound of claim 1 wherein R² and R³ are the same.

7. The compound of claim 1 wherein R³ is a hydrophobic chain.

8. The compound of claim 7 wherein the hydrophobic chain comprises an aliphatic hydrocarbon chain.

9. The compound of claim 1 wherein R¹ comprises an aromatic group.

10. The compound of claim 9 wherein the aromatic group is carbocyclic.

11. The compound of claim 1 to wherein R¹, R² and R³ are such that the compound has a critical aggregation concentration in water of below about 100 μM.

12. The compound of claim 1 which is non-cytotoxic.

13. A process for making a compound of structure (A) 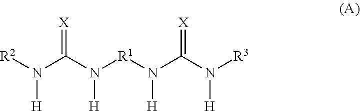
wherein:
X is either O or S;
R¹ is a rigid group;
R² is a hydrophilic group such that (A) is capable of self-assembly in water; and
R³ is an organic group,
said process comprising:
if R² and R³ are the same, reacting R¹(NCX)₂ with about two mole equivalents of R²NH₂; and
if R² and R³ are not the same, reacting R¹(NCX)₂ sequentially with R²NH₂ and R³NH₂ in either order.

14. The process of claim 13 wherein R² and R³ are not the same and the process comprises
reacting R¹(NCX)₂ with one of R²NH₂ and R³NH₂ in large molar excess of R¹(NCX)₂;
separating an intermediate adduct from excess R¹(NCX)₂; and
reacting the intermediate adduct with the other of R²NH₂ and R³NH₂ to produce the compound of structure (A).

15. A method for altering the structure of microstructures of an amphiphile in water, said amphiphile being a compound of structure (A) 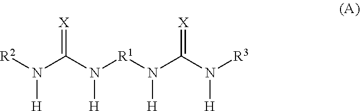
wherein:
X is either O or S;
R¹ is a rigid group;
R² is a hydrophilic group such that (A) is capable of self-assembly in water; and
R³ is an organic group,
said method comprising heating said microstructures in water to a temperature of at least about 60° C.; and cooling said heated microstructures in water to below about 40° C.

16. The method of claim 15 wherein R³ is a hydrophobic group.

17. A method for encapsulating a water soluble substance, said method comprising:
combining an aqueous solution of said substance with an amphiphile, said amphiphile being a compound of structure (A) 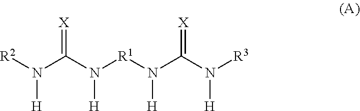
wherein:
X is either O or S;
R¹ is a rigid group;
R² is a hydrophilic group such that (A) is capable of self-assembly in water; and
R³ is an organic group; and
sonicating the resulting mixture so as to produce an aqueous product in which at least a portion of the substance is encapsulated within vesicles of the amphiphile.

18. The method of claim 17 additionally comprising dialysing the aqueous product so as to remove unencapsulated substance.

19. The method of claim 17 wherein the water soluble substance is a drug.

20. The method of claim 17 wherein the vesicles have a mean diameter of less than about 200 nm.

21. The method of claim 17 wherein R² and R³ in the amphiphile are both hydrophilic.

22. The method of claim 21 wherein R² and R³ in the amphiphile are the same.

23. A method for encapsulating a sparingly water-soluble or water insoluble substance, said method comprising:
combining a solution of said substance with an amphiphile, said amphiphile being a compound of structure (A) 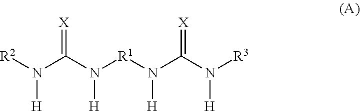
wherein:
X is either O or S;
R¹ is a rigid group;
R² is a hydrophilic group such that (A) is capable of self-assembly in water; and
R³ is an organic group
to form a mixture; and
agitating the mixture for sufficient time to form an aqueous product comprising microstructures in which the substance is encapsulated within the amphiphile.

24. The method of claim 23 additionally comprising dialysing the aqueous product so as to remove unencapsulated substance.

25. The method of claim 23 wherein the substance is a drug.

26. The method of claim 23 wherein R³ is a hydrophobic group.

27. A method comprising using a compound of structure (A) 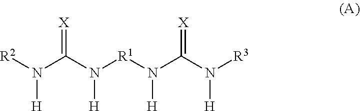
wherein:
X is either O or S;
R¹ is a rigid group;
R² is a hydrophilic group such that (A) is capable of self-assembly in water; and
R³ is an organic group
and a drug for the manufacture of a medicament for the treatment of a condition for which the drug is effective.

28. The method according to claim 27 wherein the condition is a cancer.

29. A method for treatment of a condition, said method comprising administering to the patient a therapeutically effective amount of a drug encapsulated with microstructures, said microstructures comprising an amphiphile which is a compound of structure (A) 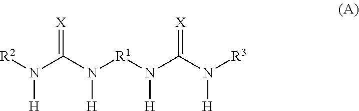
wherein:
X is either O or S;
R¹ is a rigid group;
R² is a hydrophilic group such that (A) is capable of self-assembly in water; and
R³ is an organic group
and said drug being effective for treatment of said condition.

30. A method comprising using microstructures comprising a drug encapsulated within an amphiphile in therapy, said amphiphile being a compound of structure (A) 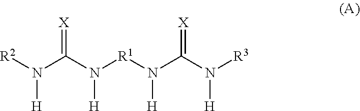
wherein:
X is either O or S;
R¹ is a rigid group;
R² is a hydrophilic group such that (A) is capable of self-assembly in water; and
R³ is an organic group.

31. A pharmaceutical composition for treatment of a condition, said composition comprising microstructures in which a drug which is effective for treatment of said composition is encapsulated within an amphiphile, said amphiphile being a compound of structure (A) 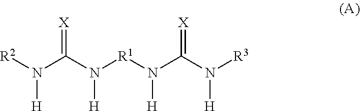
wherein:
X is either O or S;
R¹ is a rigid group;
R² is a hydrophilic group such that (A) is capable of self-assembly in water; and
R³ is an organic group,
said composition additionally comprising one or more pharmaceutically acceptable carriers, diluents and/or adjuvants.

32. A method comprising using for treatment of a condition amphiphile which is a compound of structure (A) 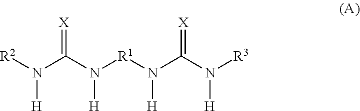
wherein:
X is either O or S;
R¹ is a rigid group;
R² is a hydrophilic group such that (A) is capable of self-assembly in water; and
R³ is an organic group
for producing microstructures.

33. The method according to claim 32 wherein the microstructures encapsulate a substance.

34. The method according to claim 33 wherein the substance is a drug.

35. The method according to claim 33 wherein the substance is sparingly water soluble or water insoluble and the microstructures are micelles or rod-like structures or emulsion droplets or platelet like structures.

36. The method according to claim 35 wherein R³ is hydrophobic.

37. The method according to claim 33 wherein the substance is water soluble and the microstructures are vesicles.

38. The method according to claim 37 wherein the R³ is hydrophihc.

39. The method according to claim 37 wherein R² and R³ are the same.

40. The method according to claim 32 wherein the microstructures are less than about 200 nm in mean diameter.

41. A process for producing microstructures, said method comprising dispersing an amphiphile which is a compound of structure (A) 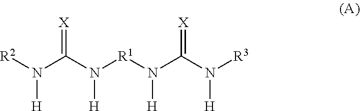
wherein:
X is either O or S;
R¹ is a rigid group;
R² is a hydrophilic group such that (A) is capable of self-assembly in water; and
R³ is an organic group
in water.

42. The process according to claim 41 wherein the microstructures encapsulate a substance.

43. The process according to claim 42 wherein the substance is a drug.

44. The process according to claim 42 wherein the substance is sparingly water soluble or water insoluble and the microstructures are micelles or rod-like structures or emulsion droplets or platelet like structures.

45. The process according to claim 44 wherein R³ is hydrophobic.

46. The process according to claim 42 wherein the substance is water soluble and the microstructures are vesicles.

47. The process according to claim 41 wherein the R³ is hydrophilic.

48. The process according to claim 46 wherein R² and R³ are the same.

49. The process according to claim 41 wherein the microstructures are less than about 200 nm in mean diameter.
